(19) United States
(12) Patent Application Publication
(10) Pub. No.: US 2025/0298009 A1
(43) Pub. Date: Sep. 25, 2025

(54) METHODS FOR CHARACTERIZING LIPID NANOPARTICLE COMPOSITIONS

(71) Applicant: Waters Technologies Corporation, Milford, MA (US)

(72) Inventors: Mateusz Imiolek, Geneva (CH); Szabolcs Fekete, Ville La Grand (FR); Matthew Lauber, North Smithfield, RI (US)

(73) Assignee: Waters Technologies Corporation, Milford, MA (US)

(21) Appl. No.: 19/086,820

(22) Filed: Mar. 21, 2025

Related U.S. Application Data
(60) Provisional application No. 63/568,544, filed on Mar. 22, 2024.

(51) Int. Cl. G01N 33/53; G01N 30/30; G01N 30/34; G01N 30/74; G01N 33/92

(52) U.S. Cl. CPC G01N 33/5308; G01N 30/30; G01N 30/34; G01N 30/74; G01N 33/92

(57) ABSTRACT

Disclosed herein are methods for characterizing lipid nanoparticles (LNPs) using size-exclusion chromatography. The disclosed methods utilize a denaturing mobile phase (i.e., denaturing SEC) to afford robust disruption of LNPs and to permit the direct injection of LNPs onto a size-exclusion chromatography column. Accordingly, said methods allow for the characterization of formulated LNPs without pre-treatment or other modifications.

### CROSS-REFERENCE TO RELATED APPLICATIONS

This application claims priority to and the benefit of U.S. Provisional Application Ser. No. 63/568,544, filed Mar. 22, 2024, and entitled “Methods for Characterizing Lipid Nanoparticle Compositions.” The contents of the foregoing application are incorporated herein by reference in their entirety.

### FIELD OF INVENTION

The present disclosure relates generally to methods for the on-line characterization of lipid nanoparticle compositions using size-exclusion chromatography.

### BACKGROUND

The use of lipid nanoparticles (LNP) for the delivery of nucleic acid payloads has increased substantially in recent years, including for the delivery of mRNA-based vaccines and gene therapies. That these therapeutics have multiple components raises several challenges and bottlenecks with respect to the measurement of critical quality attributes (CQAs), including the quantity and quality of payloads. While the detection of nucleic acids is readily achieved with UV absorption, these measurements cannot be performed on formulated LNP drug products due to the LNP shell, which introduces significant scattering of incident light at the relevant wavelengths. Alternative chromatography methods, such as reverse phase liquid chromatography (RPLC), require the use of detergents that are incompatible with RPLC columns. Accordingly, there is a need in the art for chromatographic methods that robustly and reproducibly characterize LNP drug products.

### SUMMARY OF INVENTION

The uptake of lipid nanoparticles (LNPs) as pharmaceutical drug delivery systems presents an increased need for improved methods that can robustly characterize LNPs and their respective payloads. To address these challenges, the present disclosure provides methods for the on-line characterization of formulated LNP samples using size-exclusion chromatography. Accordingly, in one aspect, disclosed herein is a method for on-line characterization of lipid nanoparticle (LNP) compositions, the method comprising: a) directly injecting onto a size-exclusion chromatography column with a formulated sample comprising an LNP, wherein the LNP comprises a lipid shell and a nucleic acid payload; b) flowing the sample through the size-exclusion chromatography column using a mobile phase comprising a detergent and an organic solvent, wherein the detergent and the organic solvent are at concentrations sufficient to denature the LNP; and c) detecting with an ultraviolet detector the lipid shell and the nucleic acid payload eluted from the column.

In some embodiments, the size-exclusion chromatography column is equilibrated with the mobile phase prior to step a). In some embodiments, the lipid shell of the LNP comprises an ionizable lipid, a phospholipid, a pegylated lipid, and/or a structural lipid. In some embodiments, the nucleic acid payload, is mRNA, guide RNA, and/or small interfering RNA (siRNA).

In some embodiments, the detergent is an ionic detergent or a non-ionic detergent. In some embodiments, the ionic detergent is sodium dodecyl sulfate (SDS), sodium lauroyl sarcosinate (sarcosyl), sodium deoxycholate, or sodium cholate. In some embodiments, the non-ionic detergent is polyethylene glycol p-(1,1,3,3-tetramethylbutyl)-phenyl ether also referred to as polyethylene glycol tert-octylphenyl ether (e.g., sold under trade name Triton™-X-100 available from Dow Chemical Company), digitonin, polysorbate, such as polysorbate 20 or polysorbate 80 (such as Tween®-20, or Tween®-80 available from Croda Americas LLC). In some embodiments, the detergent is SDS. In some embodiments, the concentration of the detergent is about 0.1% to about 1.0% (w/v).

In some embodiments, the organic solvent is isopropyl alcohol, ethanol, methanol, acetonitrile, butanol, or a combination thereof. In some embodiments, the organic solvent is isopropyl alcohol. In some embodiments, the concentration of the isopropyl alcohol is about 5% to about 40% (v/v).

In some embodiments, the detergent used in the mobile phase is SDS and the organic solvent is isopropyl alcohol. In some embodiments, the concentration of the SDS is about 0.1% to about 1.0% (w/v) and the concentration of the isopropyl alcohol is about 5% to about 40% (v/v). In some embodiments, the concentration of the SDS is about 0.2% (w/v) and the concentration of the isopropyl alcohol is about 20% (v/v).

In some embodiments, the method is performed at a temperature of between about 25° C. to about 55° C.

In some embodiments, the size-exclusion chromatography column comprises diol-bonded porous particles having a particle size of between 1 μm to 10 μm. In some embodiments, the size-exclusion chromatography column comprises diol-bonded porous particles having an average pore diameter of between 100 Å to 5000 Å.

In some embodiments, the size exclusion chromatography column comprises a chromatography material comprising porous silica particles having a particle size of between 1 μm to 10 μm, wherein the silica particles comprise a modified surface. In some embodiments, the modified surface comprises a bridged ethylene polyethylene hydroxide surface. In some embodiments, the modified surface comprises an ethylene polyethylene methoxide surface.

In some embodiments, the mobile phase further comprises a buffer. In some embodiments, the buffer is phosphate-buffered saline (PBS).

In some embodiments, the ultraviolet detector of step c) measures at between 210 nm to 300 nm. In some embodiments, the ultraviolet detector of step c) measures at between 260 nm to 280 nm.

### BRIEF DESCRIPTION OF THE DRAWINGS

The technology will be more fully understood from the following detailed description taken in conjunction with the accompanying drawings.

FIG. 1 is a graphical illustration of a method of characterizing a formulated LNP sample according to some embodiments of the present technology.

FIG. 2A-2B provide chromatograms of LNPs (FIG. 2A) or mRNA isolated from LNPs (FIG. 2B) using non-denaturing SEC.

FIG. 3A-3C provide 230 nm (230) and 260 nm (260) traces for pre-treated LNP samples using non-denaturing SEC. FIG. 3A shows the Moderna LNP sample. FIG. 3B shows the Pfizer LNP sample. FIG. 3C shows the PackGene LNP sample.

FIG. 4A-4D provide 230 nm (230) and 260 nm (260) traces for LNP samples using denaturing SEC. FIG. 4A shows the Moderna LNP sample. FIG. 4B shows the Pfizer LNP sample. FIG. 4C shows the PackGene LNP sample. FIG. 4 D shows a control SpCas9 mRNA sample.

FIG. 5 provides an overlay of the 260 nm chromatograms of Moderna LNP (Moderna trace) and PackGene LNP (PackGene trace) using denaturing SEC.

FIG. 6A demonstrates the signal response by 260 nm peak area for the Pfizer LNP sample and a Cas9 mRNA control using denaturing or non-denaturing SEC.

FIG. 6B shows the linear response between 260 nm peak area and injection volume for a Pfizer LNP sample.

FIG. 7A shows a chromatogram demonstrating the separation of the Moderna COVID-19 vaccine (NDC 80777-279-99), the Pfizer COVID-19 vaccine (NDC 59267-1025-4), and the PackGene Biotechnology FireFly Luciferase-mRNA LNP using 3 μm particles including 1000 Å pores and a bridged ethylene polyethylene oxide hydroxyl terminated surface.

FIG. 7B shows a chromatogram demonstrating the separation of the Moderna COVID-19 vaccine (NDC 80777-279-99), the Pfizer COVID-19 vaccine (NDC 59267-1025-4), and the PackGene Biotechnology FireFly Luciferase-mRNA LNP using 3.3 μm particles including 1000 Å pores and a polyethylene oxide methoxide terminated surface.

FIG. 7C shows a chromatogram demonstrating the separation of the Moderna COVID-19 vaccine (NDC 80777-279-99), the Pfizer COVID-19 vaccine (NDC 59267-1025-4), and the PackGene Biotechnology FireFly Luciferase-mRNA LNP using 3.3 μm particles including 2000 Å pores and a polyethylene oxide hydroxide terminated surface.

### DETAILED DESCRIPTION

Disclosed herein are methods for characterizing formulated LNP samples using size-exclusion chromatography. In order that the technology may be more readily understood, certain terms are first defined. It should be noted that whenever a value or range of values of a parameter are recited, it is intended that values and ranges intermediate to the recited values are part of this disclosure. The word “about” if not otherwise defined means±5%. It is also to be noted that as used herein and in the claims, singular forms of “a”, “and”, and “the” include plural references unless the context clearly dictates otherwise.

### Definitions

As used herein, the term “lipid nanoparticle” or “LNP” refers to a nanoparticle composed of lipids and a nucleic acid payload. A lipid nanoparticle may comprise one or more types of lipids, including ionizable lipids (such as ionizable cationic lipids), phospholipids, structural lipids (such as cholesterol), and pegylated lipids. Lipids suitable for use in lipid nanoparticle compositions are further described in U.S. Pat. No. 11,786,607, PCT publication WO 2017/223,135, and U.S. Pat. No. 10,507,249 each of which are incorporated herein by reference.

As used herein, the terms “formulated LNP,” “formulated LNP sample,” or “formulated sample” refer to a sample that has not undergone pre-treatment prior to an analytical assay, such as size exclusion chromatography. Examples of pre-treatment include, but not are limited to, the addition of a solvent, detergent, or other compound to the sample prior to analysis. In one aspect of the present disclosure, a formulated LNP sample is directly injected onto a size-exclusion chromatography column. As used herein “directly injected” refers to a sample being injected onto a size-exclusion chromatography column without any intermediate pre-treatment.

As used herein, the term “denaturing size-exclusion chromatography” refers to size-exclusion chromatography performed using a mobile phase that results in denaturing of lipid nanoparticles. In some embodiments, the mobile phase comprises a detergent and an organic solvent.

### Methods of Lipid Nanoparticle Characterization

Disclosed herein are methods of characterizing formulated lipid nanoparticle compositions using size-exclusion chromatography (SEC). Prior efforts, which have utilized positively charged resins, have faltered due to the adsorption and incomplete elution of nucleic acids, leading to a lack of reproducibility across batch analyses. To address this issue, the present disclosure utilizes low adsorption SEC columns in conjunction with a mobile phase that comprises both a detergent and an organic solvent (i.e., a denaturing mobile phase). The denaturing mobile phase comprising a detergent and co-solvent is important to ensure complete disruption of the LNP shell. Notably, it was found that using the mobile phase comprising the detergent and an organic solvent was sufficient to fully disrupt LNPs in an on-line (i.e., on-column) method. As such, the presently disclosed methods allow for the analysis of formulated LNP samples without any pre-treatment or modifications otherwise. That is, this method allows for direct injection of samples onto an SEC column, simplifying workflows and removing potential sample manipulation variability

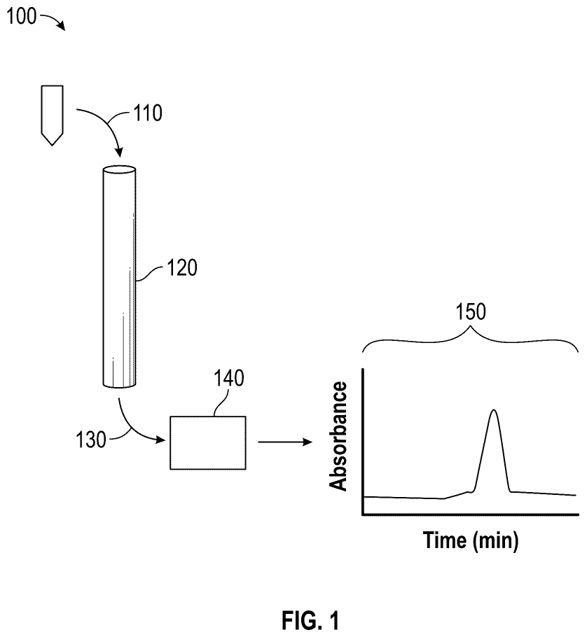

FIG. 1 illustrates an embodiment of the present disclosure. Formulated LNP sample (100) is directly injected (110) onto an SEC column (120). The sample is eluted using a mobile phase comprising an organic solvent and detergent. The eluted sample (130) is detected using an ultraviolet (UV) detector (140), resulting in a chromatogram (150). Due to the complete disruption of the LNP sample, the disclosed methods afford robust quantification of nucleic acid payloads via UV absorbance. The methods further allow for the detection of differentially sized nucleic acid payloads, including impurities or degradation products, that would not otherwise be detected in other chromatography methods (i.e., RPLC) or non-chromatographic quantification methods (such as fluorimetry, see e.g., RiboGreen® RNA fluorescence stains). In some embodiments, UV detector (140) is an alternative detector such as a fluorescence or mass spectrometry detector as described below. In some embodiments, UV detector (140) is connected in series to one or more additional detectors.

To aid in quantitation, reference nucleic acid material (such as mRNA) may be utilized. Reference material may be obtained using methods known in the art, including alcohol-mediated precipitation. For example, an LNP sample may be diluted in an alcohol which precipitates the nucleic acids as described in Example 1. The precipitated material can be washed and re-suspended in aqueous buffer for downstream use, for example to determine the retention time of the nucleic acids of interest.

### Denaturing Mobile Phase Compositions

The presently disclosed methods utilize a mobile phase that comprises a detergent and an organic solvent in concentrations sufficient to afford complete disruption of LNPs. A number of organic solvents are suitable for use, including isopropyl alcohol (also referred to as isopropanol interchangeably), ethanol, methanol, acetonitrile, butanol, or a combination thereof. In a preferred embodiment, the organic solvent is isopropyl alcohol.

The organic solvent may be present in the mobile phase at a concentration of about 5% to about 40% (v/v). In some embodiments, the organic solvent is present in the mobile phase at a concentration of about 5-10% (v/v), 10-15% (v/v), 15-20% (v/V), 20-25% (v/v), 25-30% (v/v), 30-35% (v/v), or 35-40% (v/v). In some embodiments, the organic solvent is present at 20% (v/v). In some embodiments, the organic solvent is isopropyl alcohol at a concentration of about 5-40% (v/v). In a preferred embodiment, the solvent is isopropyl alcohol at a concentration of about 20% (v/v).

A number of detergents are suitable for use in the mobile phase, including ionic detergents and non-ionic detergents. In some embodiments, the ionic detergent is sodium dodecyl sulfate (SDS), sodium lauroyl sarcosinate (sarcosyl), sodium deoxycholate, or sodium cholate). In some embodiments, the non-ionic detergent is polyethylene glycol tert-octylphenyl ether (commercially available from Dow Chemical and associated with the trademark Triton™-X-100), digitonin, polysorbate 20 (associated with the trademark Tween®-20), or polysorbate 80 (associated with the trademark Tween®-80). In a preferred embodiment, the detergent is SDS. While the detergent must be sufficient to disrupt the LNP, it is further beneficial that the detergent does not absorb at the measured wavelengths, such as between 230-260 nm wavelengths.

The detergent may be present in the mobile phase at a concentration of about 0.1% to about 1.0% (w/w). In some embodiments, the detergent is present in the mobile phase at a concentration of about 0.1-0.2% (w/v), 0.2-0.3% (w/v), 0.3-0.4% (w/v), 0.4-0.5% (w/v), 0.5-0.6% (w/v), 0.6-0.7% (w/v), 0.7-0.8% (w/v), 0.8-0.9% (w/v), or 0.9-1.0% (w/v). In some embodiments, the detergent is present at 0.2% (w/v). In some embodiments, the detergent is SDS at a concentration of 0.1-1.0% (w/v). In a preferred embodiment, the detergent is SDS at a concentration of about 0.2% (w/v).

In some embodiments, the mobile phase consists essentially of a detergent and an organic solvent. In some embodiments, the mobile phase consists essential of SDS and isopropyl alcohol. In some embodiments, the SDS of said mobile phase is present at a concentration of about 0.1-1.0% and the isopropyl alcohol at a concentration of about 5-40%.

The mobile phase may further comprise a buffer. It is important to note that, while the buffer is important for the stability of the nucleic acids, it does not contribute to the denaturing capacity of the mobile phase (i.e., the buffer does not contribute to the denaturation of the LNP itself). Accordingly, in some embodiments the mobile phase further comprises a buffer, such as, for example, phosphate-buffered saline (PBS). In some embodiments, the PBS is present at 1× concentration or at 2× concentration. Other buffers suitable for use with nucleic acid molecules are known and readily understood by one of ordinary skill in the art.

### Size-Exclusion Chromatography Columns and Detectors

A number of size-exclusion chromatography columns, sizes, and materials are suitable for use in the methods disclosed herein.

In some embodiments, the size-exclusion chromatography column comprises particles having diameters ranging from between 1 to 10 μm, more preferably 2 to 7 μm. In some embodiments, the particles have an average diameter of about 2.5 μm.

In some embodiments, the size-exclusion chromatography column comprises particles having an average pore diameter ranging from between 100 to 5000 Å, more preferably 200 to 3000 Å. In some embodiments, the particles have an average pore diameter of about 450 Å.

Particle compositions suitable for use include, but are not limited to, inorganic materials (e.g., silica), organic material, or hybrid inorganic/organic material. Examples of suitable particle compositions are further described in US Patent Application No. 2021/0239655, U.S. Pat. Nos. 11,478,755 11,426,707, and 7,919,177, each of which are incorporated by reference.

A particle described herein may include one or more ethylene groups bridging between silicon atoms in (hereafter referred to as “bridging ethylene” or BE groups). In some embodiments, BE groups are only present on the surface of a particle.

In some embodiments, the exterior surface of the particles may be modified or functionalized to change the chemical interaction (e.g., electrostatic interaction) between the chromatography material and the LNP. For example, particles may be functionalized to include an alkoxy functionalized surface or a hydroxy functionalized surface. A surface modification may be introduced by interacting a particle with an organic material (e.g., polyethylene oxide (PEO)) terminated with an alkoxy group or a hydroxy group.

A particle with a surface modification can be formed by interacting that particle with PEO. In some embodiments, the PEO may include hydroxy groups (e.g., PEO hydroxide). In some embodiments, the PEO may include methoxy groups (e.g., PEO methoxide). In some embodiments, the hydroxy or methoxy group may be introduced to the PEO after interacting the particle with PEO. In some embodiments, the hydroxy or methoxy groups may be included in the PEO prior to interacting the particle with the PEO. In some embodiments, the modified surface comprises a bridged ethylene polyethylene hydroxide surface. In some embodiments, the modified surface comprises an ethylene polyethylene methoxide surface.

Particles including a surface modification or functionalization and methods of preparation thereof are known in the art, e.g., U.S. Pat. Nos. 7,396,468; 7,846,337; 7,943,046; 8,864,988; 8,828,904; 9,284,456; 10,618,920; 10,092,893; and U.S. Patent Publication No. US 2022/0080385; the surface functionalizations and surface modifications of which are incorporated here by reference.

In some embodiments, the particle is a BEH (bridged ethylene hybrid) particle comprising tetraethoxysilane (TEOS) and bis(triethoxysilyl)ethane (BTEE). In some embodiments, the particle is a porous, diol-bonded BEH particle. In some embodiments, the particle is a diol-bonded silica particle. In some embodiments, the particle is a silica particle with a methoxy-terminated polyethylene oxide (PEO) bonding. In some embodiments, the particle is a methacrylate and polymer bead type.

The column material may be stainless steel, polyetheretherketone (PEEK) lined steel, titanium, or a stainless alloy. Column inner diameters may range from about 2.1 mm to about 7.8 mm. Column lengths may range from about 10 mm to about 300 mm. Exemplary column dimensions include, but are not limited to, 2.1×20 mm, 2.1×50 mm, 2.1×100 mm, 2.1×150 mm, 4.6×50 mm, 4.6×100 mm, 4.6×150 mm, and 4.6×300 mm.

The choice of column and particle, particularly with respect to pore diameter and particle size, is in part dependent on the size of the molecule to be detected and separated as would be appreciated and readily understood by one of ordinary skill in the art.

Chromatography columns suitable for use with the methods disclosed herein are compatible with any liquid chromatography system, including high-performance liquid chromatography (HPLC) and ultra-high performance liquid chromatography (UHPLC) systems, including UPLC™ systems available from Waters Corporation.

According to embodiments of the present disclosure, the size-exclusion chromatography columns are connected in fluidic series to a detector. In one aspect, the detector is an ultraviolet (UV) detector or a tunable UV (TUV) detector. In some embodiments, the UV or TUV detector measures at between 210 nm to 300 nm. In preferred embodiments, the UV or TUV detector measures at between 230 to 260 nm, or more preferably at 230 nm and 260 nm. Said wavelengths are known in the art to detect nucleic acid molecules, including RNA. Additional detectors, such as fluorescence spectroscopy or mass spectrometry detectors can be utilized in conjunction with the disclosed methods. The detectors can be used alone or in tandem and can be further adjusted to detect molecule(s) of interest. For example, and not by way of limitation, a fluorescence detector may be utilized if the sample comprises a fluorescent molecule of interest.

In some embodiments, the interior surfaces of the column are treated to reduce non-specific binding and enhance overall efficiency of the size-exclusion chromatography system. In particular, an alkylsilyl coating or other high performance surface is provided to limit or reduce non-specific binding of a sample with walls or interior surfaces of a column body. Without wishing to be bound by theory, it is believed that an alkylsilyl coating covering metal surfaces prevents or minimizes contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating comprises a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. Other embodiments of alkylsilyl coatings suitable for use with the present technology are described in U.S. Patent Publication No. 2019/0086371 and U.S. Application Publication No. 2022/0118443 (which are hereby incorporated by reference).

### EXAMPLES

### Example 1: Analysis of LNPs Using Non-Denaturing or Denaturing SEC

LNP samples were assessed using three methods: a) non-denaturing SEC; b) sample pre-treatment with non-denaturing SEC; or c) no sample pre-treatment with denaturing SEC (i.e., mobile phase including both organic solvent and detergent). Commercially available LNP sources were utilized, including an LNP sample purchased from Moderna (COVID-19 vaccine; NDC 80777-279-99), Pfizer (COVID-19 vaccine; NDC 59267-1025-4), and PackGene Biotechnology (FireFly Luciferase-mRNA LNP). eSpCas9 mRNA purchased from GenScript was used as a reference material.

All experiments were performed using an ACQUITY™ UPLC™ H-Class Bio QSM System (available from Waters Corporation) equipped with a tunable UV detector. This system used a 4.6×150 mm SEC column packed with 2.5 μm diol-bonded BEH particles with 450 Å pore size (XBridge™ Premier Gtx BEH SEC column available from Waters Corporation).

As controls, mRNA was isolated from respective LNP samples using alcohol mediated precipitation. Briefly, 50 μL of an LNP sample was diluted with 1 mL of 60 mM NH₄OAc in 100% isopropanol (IPA). Samples were centrifuged at 14,000×g at 4° C. for 15 minutes, after which the supernatant was removed and the pellet washed with 100% IPA. Samples were centrifuged under the same conditions, supernatant removed, and the pellet of mRNA dried under N₂. The pellet was re-suspended in 50 μL of water.

### Non-Denaturing SEC

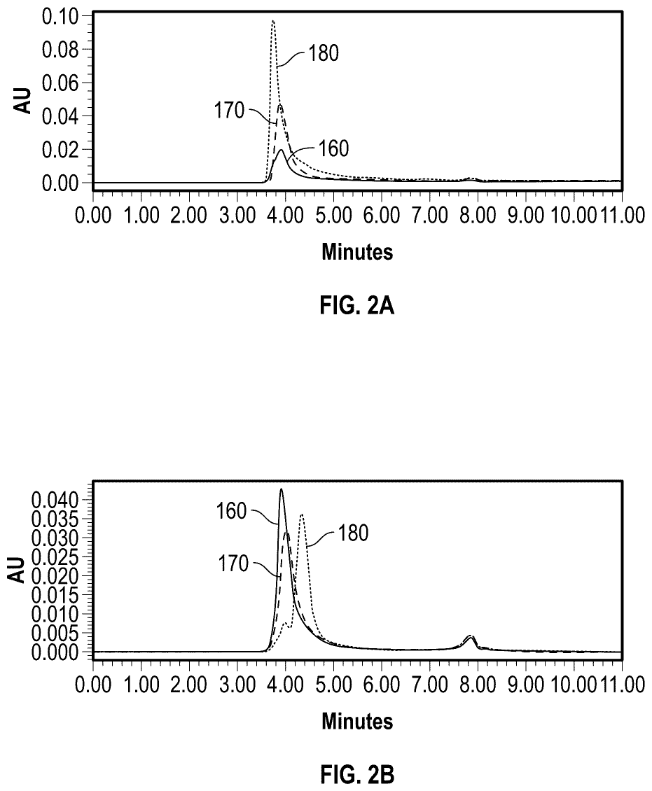

LNP samples were first analyzed using non-denaturing SEC. LNP samples were run on the SEC system using 1× phosphate-buffered saline (PBS) buffer at ambient temperature (˜25° C.). The three LNP samples, Moderna (labeled 160 trace) and having ˜80 nm LNP size, Pfizer (labeled 170 trace) and also having ˜80 nm LNP size, and PackGene (labeled 180 trace) and having ˜100 nm LNP size), eluted at similar times (FIG. 2A). This indicated that the LNP samples were not disrupted during the SEC analysis. To confirm this, mRNA isolated from the respective samples was run on the same column, which showed separation of the PackGene mRNA (˜2k base pairs) from the Moderna and Pfizer mRNA (˜5k base pairs) as shown in FIG. 2B. A summary of said results, including the A_{260/230} ratio (a metric of RNA purity; with a value of 1.8-2.2 indicative of pure RNA) is shown in Table 1.

TABLE 1
Parameters of LNP Samples from Native, Non-Denaturing SEC

| | Elution Time | |
|---|---|---|
| Sample | (min) | A_{260/230} Ratio |
| Pfizer LNP | 3.89 | 0.84 |
| Pfizer mRNA | 3.95 | 2.08 |
| Moderna LNP | 3.91 | 0.93 |
| Moderna mRNA | 4.03 | 1.99 |
| PackGene LNP | 3.75 | 0.59 |
| PackGene mRNA | 4.36 | 2.04 |
| eSpCas9 mRNA | 3.87 | 2.14 |

Sample Pre-Treatment with Non-Denaturing SEC

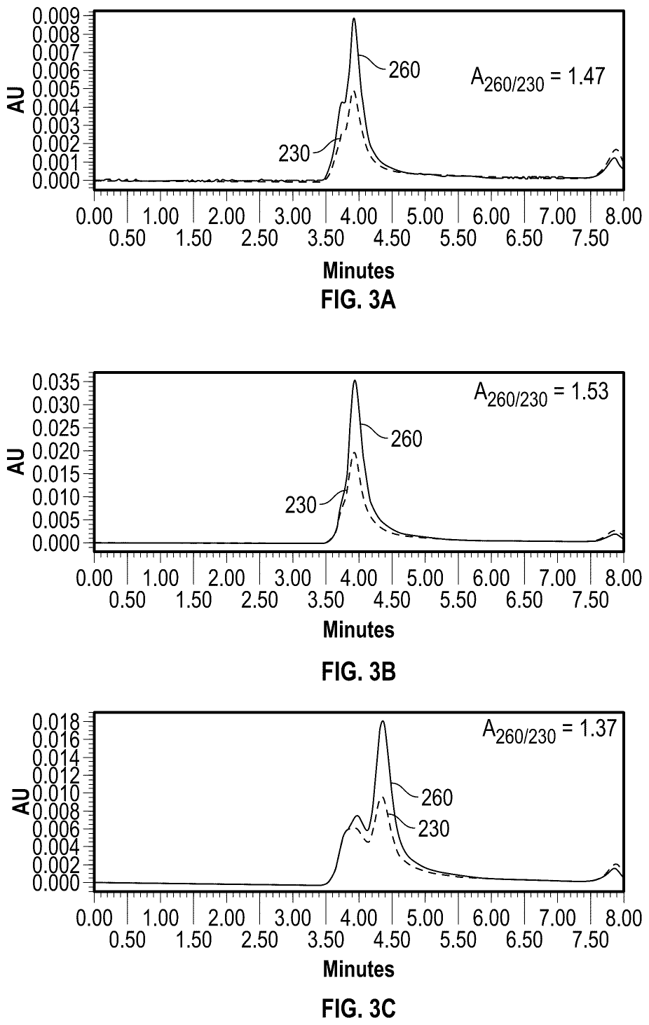

It was next determined if pre-treatment of LNP samples with a denaturing agent (e.g., a detergent) was sufficient for analysis with non-denaturing SEC. Samples were pre-treated with 0.1% Triton™ X-100, a non-ionic surfactant, at 25° C. for 15 minutes with pipette mixing prior to being injected onto the SEC column. The tested pre-treatment condition was not sufficient to achieve complete disruption of the LNP samples as indicated by the low A_{260/230} ratios. FIG. 3A shows the 260 nm trace (260) and 230 nm trace (230) for the Moderna sample; FIG. 3B shows the 260 nm trace (260) and 230 nm trace (230) for the Pfizer sample; and FIG. 3C shows the 260 nm trace (260) and 230 nm trace (230) for the PackGene sample. A summary of the A_{260/230} ratios for each sample are provided in Table 2. Additional conditions were tested and did not yield sufficient disruption of the LNPs (data not shown). These included incubation at higher temperatures (1-15 minutes; 60-90° C.), Triton™ X-100 concentrations up to 1% (w/v), SDS concentrations up to 1% (w/v), pluronic F168 concentrations up to 5%, dilution in 5% acetonitrile, dilution in isopropanol up to 40%, dilution in DMSO up to 40%, salt concentrations up to 1M NaCl, acidic conditions (pH 4.0), and alkaline conditions (pH 11.0).

TABLE 2
A_{260/230} Ratios of Pre-Treated Samples from Non-Denaturing SEC

| Sample | A_{260/230} Ratio |
|---|---|
| Moderna LNP | 1.47 |
| Pfizer LNP | 1.53 |
| PackGene LNP | 1.37 |

No Sample Pre-Treatment with Denaturing SEC

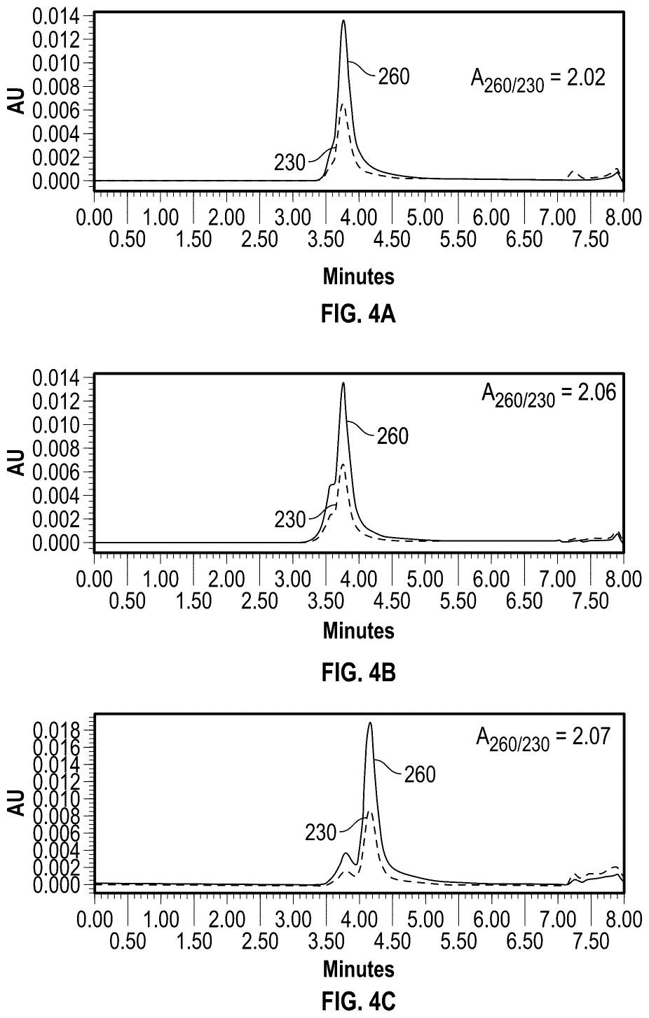

The use of a denaturing mobile phase was tested to determine if said conditions afforded sufficient disruption of LNP samples. LNP samples were injected onto the SEC column and flowed with a mobile phase comprising 1×PBS, 0.2% sodium dodecyl sulfate (SDS) and 20% isopropanol at 40° C. Said conditions resulted in robust disruption of the tested LNP samples as evidenced by the A_{260/230} ratios of ˜2. FIG. 4A shows the 260 nm trace (260) and 230 nm trace (230) for the Moderna sample; FIG. 4B shows the 260 nm trace (260) and 230 nm trace (230) for the Pfizer sample; FIG. 4C shows the 260 nm trace (260) and 230 nm trace (230) for the PackGene sample; and FIG. 4D shows the 260 nm trace (260) and 230 nm trace (230) for a control SpCas9 mRNA sample. A summary of the A_{260/230} ratios for each sample are provided in Table 3. Comparable results were obtained using a mobile phase comprising 1.0% SDS at 55° C. (data not shown).

TABLE 3
A_{260/230} Ratios of Samples from Denaturing SEC Ratio

| Sample | A_{260/230} Ratio |
|---|---|
| Moderna LNP | 2.02 |
| Pfizer LNP | 2.06 |
| PackGene LNP | 2.06 |
| SpCas9 mRNA | 2.00 |

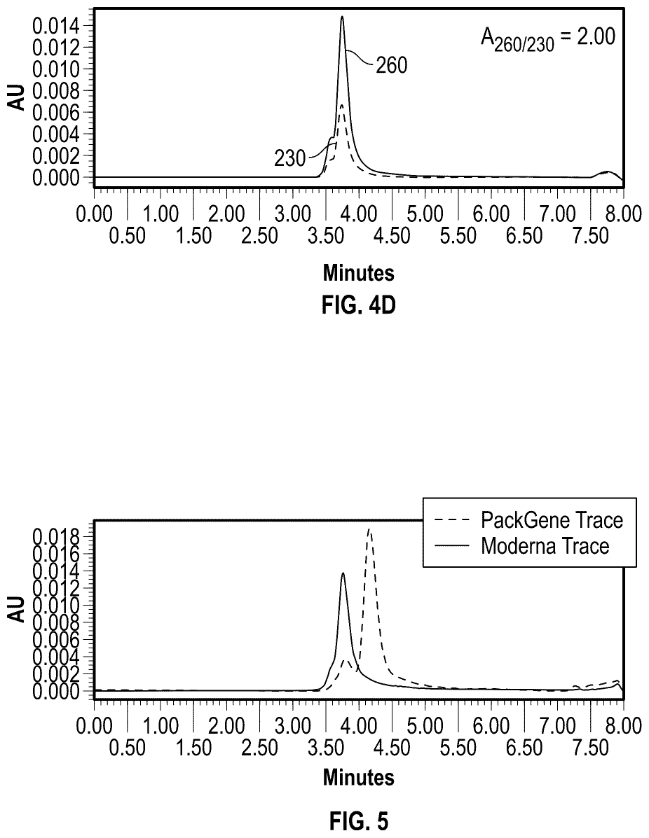

Notably, the denaturing SEC method further afforded separation of different size mRNA payloads, such as the mRNA of the Moderna LNP (Moderna trace) and the mRNA of the PackGene LNP (PackGene trace) (FIG. 5).

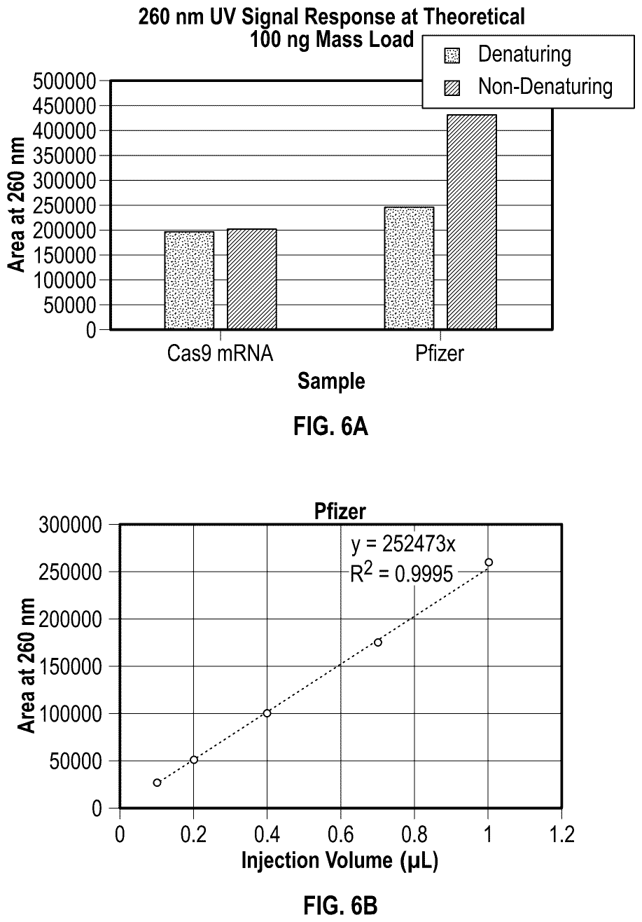

The denaturing SEC method further allows for quantification of mRNA when used in conjunction with a reference mRNA at a known concentration. FIG. 6A shows the analysis of absolute peak areas at 260 nm for the different LNP samples and reference mRNA for denaturing SEC (left hand box of data set) or non-denaturing SEC (right hand box of data set). The use of non-denaturing SEC resulted in larger than expected response (scattered light at 260 nm wavelength) as compared to a control sample of Cas9 mRNA (similar size) at the same mass load. In contrast, denaturing SEC conditions resulted in absolute peak areas within the expected range as compared to the same control same.

FIG. 6B demonstrates the linearity of detector response across different injection volumes for the Pfizer LNP sample.

In summary, the denaturing SEC method afforded robust disruption of multiple LNP samples, allowing for the characterization of LNP formulations in the absence of any pre-treatment conditions. This method further allows for direct injection of samples onto an SEC column, simplifying workflows and removing potential sample manipulation variability.

### Example 2: Denaturing SEC Using Particles with Modified Surfaces

The effect of particle surface chemistry on denaturing SEC was then examined. The present Example is directed to the investigation of three different particle chemistries together with Applicant's claimed method of direct injection using a denaturing mobile phase. Three commercially available LNP samples were used in the analysis of particle chemistry. The LNPs were: the Moderna COVID-19 vaccine (NDC 80777-279-99), the Pfizer COVID-19 vaccine (NDC 59267-1025-4), and the PackGene Biotechnology FireFly Luciferase-mRNA LNP. Samples were prepared as described in Example 1. All experiments were performed using a high performance liquid chromatography system, specifically ACQUITY™ UPLC™ H-Class Bio QSM System (available from Waters Corporation), equipped with a tunable UV detector. Each LNP sample was directly injected onto a column with the investigated particle chemistry using a mobile phase comprising 1×PBS, 0.2% sodium dodecyl sulfate (SDS) and 20% isopropanol at 40° C.

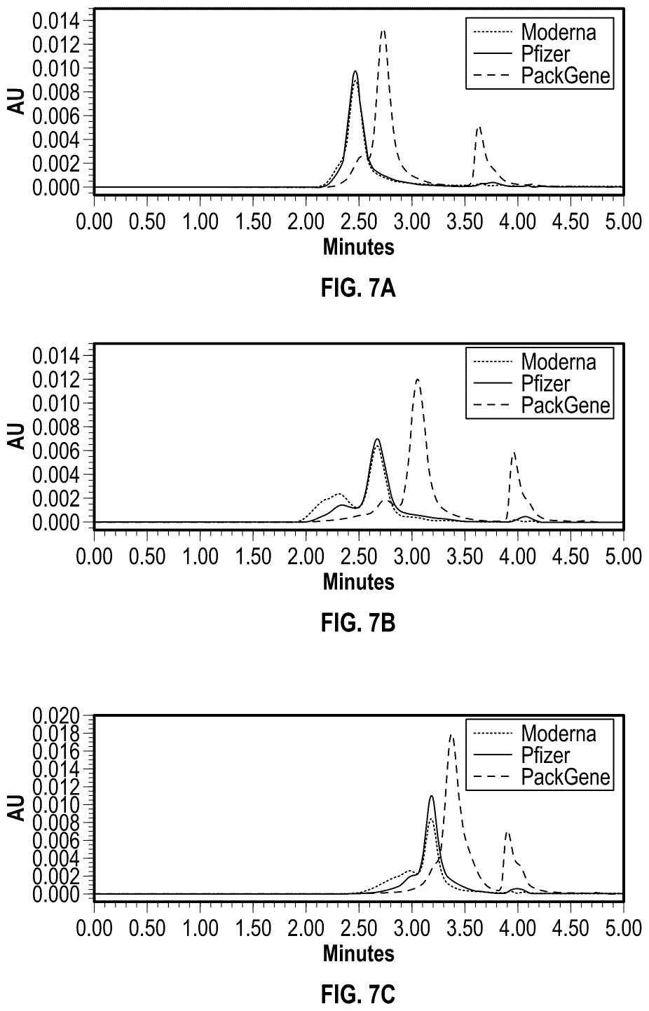

In a first experiment, a chromatography material composed of 3 μm silica particles with 1000 Å pores and bridged ethylene hydroxide-terminated PEO modified surfaces was utilized in an SEC experiment. The result of the SEC experiment was characterized by the A_{260/230} ratio (a metric of RNA purity; with a value of 1.8-2.2 indicative of pure RNA) of the RNA payload. The results are summarized in FIG. 7A, with A_{260/230} values for the three LNP samples summarized in Table 4.

TABLE 4
A_{260/230} Ratios of Samples from 2.7 μm Hydroxide-
terminated PEO Surface Particles

| Sample | A_{260/230} Ratio |
|---|---|
| Moderna LNP | 2.16 |
| Pfizer LNP | 2.14 |
| PackGene LNP | 2.07 |

In the second experiment, a chromatography material composed of 2.7 μm silica particles with 1000 Å pores and polyethylene oxide methoxide modified surfaces was utilized in an SEC experiment. The result of the SEC experiment was characterized by the A_{260/230} ratio (a metric of RNA purity; with a value of 1.8-2.2 indicative of pure RNA) of the RNA payload. The results are summarized in FIG. 7B, with A_{260/230} values for the three LNP samples summarized in Table 5.

TABLE 5
A_{260/230} Ratios of Samples from 3.0 μm Methoxide-
terminated PEO Surface Particles

| Sample | A_{260/230} Ratio |
|---|---|
| Moderna LNP | 2.11 |
| Pfizer LNP | 2.12 |
| PackGene LNP | 2.11 |

In a third experiment, a chromatography material composed of 3.3 μm silica particles with 2000 Å pores and bridged ethylene hydroxide-terminated polyethylene oxide modified surfaces were utilized in a chromatography experiment. The result of the SEC experiment was characterized by the A_{260/230} ratio (a metric of RNA purity; with a value of 1.8-2.2 indicative of pure RNA) of the RNA payload. The results are summarized in FIG. 7B, with A_{260/230} values for the three LNP samples summarized in Table 6.

TABLE 6
A_{260/230} Ratios of Samples from 3.3 μm Hydroxide-
terminated PEO Surface Particles

| Sample | A_{260/230} Ratio |
|---|---|
| Moderna LNP | 2.11 |
| Pfizer LNP | 2.08 |
| PackGene LNP | 2.11 |

In each experiment, the resulting RNA A_{260/230} value was in the ideal 1.8-2.2 range, indicating high purity RNA was liberated from the LNP in each experiment. Additionally, each experiment demonstrated the ability to separate the aggregated forms of the RNA payload during the experiment. Moreover, separation of aggregated forms of the large RNA payloads was observed to be more efficient in chromatography experiments using wider pore particles.

1. A method for on-line characterization of lipid nanoparticle (LNP) compositions, the method comprising:
a) directly injecting onto a size-exclusion chromatography column with a formulated sample comprising an LNP, wherein the LNP comprises a lipid shell and a nucleic acid payload;
b) flowing the sample through the size-exclusion chromatography column using a mobile phase comprising a detergent and an organic solvent, wherein the detergent and the organic solvent are at concentrations sufficient to denature the LNP; and
c) detecting with an ultraviolet detector the lipid shell and the nucleic acid payload eluted from the column.

2. The method of claim 1, wherein the size-exclusion chromatography column is equilibrated with the mobile phase prior to step a).

3. The method of claim 1, wherein the lipid shell comprises an ionizable lipid, a phospholipid, a pegylated lipid, and/or a structural lipid.

4. The method of claim 1, wherein the nucleic acid payload is mRNA, guide RNA (gRNA), and/or small interfering RNA (siRNA).

5. The method of claim 1, wherein the detergent is an ionic detergent or a non-ionic detergent.

6. The method of claim 5, wherein the ionic detergent is sodium dodecyl sulfate, sodium lauroyl sarcosinate, sodium deoxycholate, or sodium cholate.

7. The method of claim 5, wherein the non-ionic detergent is polyethylene glycol tert-octylphenyl ether, digitonin, polysorbate 20, or polysorbate 80.

8. The method of claim 6, wherein the detergent is sodium dodecyl sulfate.

9. The method of claim 1, wherein the concentration of the detergent is about 0.1% to about 1.0% (w/v).

10. The method of claim 1, wherein the organic solvent is isopropyl alcohol, ethanol, methanol, acetonitrile, butanol, or a combination thereof.

11. (canceled)

12. The method of claim 1, wherein the concentration of the organic solvent is about 5% to about 40% (v/v).

13. The method of claim 1, wherein the detergent is sodium dodecyl sulfate and the organic solvent is isopropyl alcohol.

14. The method of claim 13, wherein the concentration of the sodium dodecyl sulfate is about 0.1% to about 1.0% (w/v) and the concentration of the isopropyl alcohol is about 5% to about 40%.

15. (canceled)

16. The method of claim 1, wherein the method is performed at a temperature of between about 25° C. to about 55° C.

17. The method of claim 1, wherein the size-exclusion chromatography column comprises diol-bonded porous particles having a particle size of between 1 μm to 10 μm.

18. The method of claim 1, wherein the size-exclusion chromatography column comprises porous silica particles having a particle size of between 1 μm to 10 μm, wherein the silica particles comprise a modified surface.

19. The method of claim 18, wherein the modified surface comprises a bridged ethylene polyethylene hydroxide terminated surface.

20. The method of claim 18, wherein the modified surface comprises an ethylene polyethylene methoxide terminated surface.

21. The method of claim 1, wherein the size exclusion chromatography column comprises:
diol-bonded porous particles having an average pore diameter of between 100 Å to 5000 Å; or
porous silica particles having an average pore diameter of between 100 Å to 5000 Å.

22. The method of claim 1, wherein the mobile phase further comprises a buffer, wherein the buffer is phosphate-buffered saline.

23. (canceled)

24. The method of claim 1, wherein the ultraviolet detector of step c) measures at between 210 nm to 300 nm.

25. (canceled)
